(19) United States
(12) Patent Application Publication
(10) Pub. No.: US 2016/0000974 A1
(43) Pub. Date: Jan. 7, 2016

(54) Composite Matrix for Bone Repair Applications

(71) Applicants: Treena L. Arinzeh, West Orange, NJ (US); Cheul Hyung Cho, Whippany, NJ (US); Saranya Elavazhagan, Westerville, OH (US)

(72) Inventors: Treena L. Arinzeh, West Orange, NJ (US); Cheul Hyung Cho, Whippany, NJ (US); Saranya Elavazhagan, Westerville, OH (US)

(73) Assignee: NEW JERSEY INSTITUTE OF TECHNOLOTY, Newark, NJ (US)

(21) Appl. No.: 14/381,496

(22) PCT Filed: Aug. 9, 2012
(86) PCT No.: PCT/US12/50156

§ 371 Date: Jul. 21, 2015

Related U.S. Application Data
(60) Provisional application No. 61/521,456, filed on Aug. 9, 2011.

(51) Int. Cl. A61L 27/58; A61L 27/38; A61L 27/56; A61L 27/36; A61L 27/18; A61L 27/12; A61L 27/54; A61L 27/46

(52) U.S. Cl. CPC A61L 27/58; A61L 27/54; A61L 27/3847; A61L 27/3834; A61L 27/46; A61L 27/365; A61L 27/18; A61L 27/12; A61L 27/3608; A61L 27/56; A61L 2430/02; A61L 2300/64; A61L 2300/604; A61L 2300/412; A61L 2400/12

(57) ABSTRACT

Composite fibrous and non-fibrous matrices of biocompatible, bioactive synthetic polymers and ceramics are described. The composite matrices support bone cell differentiation and may be used alone or with whole bone marrow, isolated mesenchymal stem cells and/or bone grafts for bone repair and bone regeneration.

### RELATED APPLICATIONS

This application claims priority to U.S. Provisional Application No. 61/521,456, filed on Aug. 9, 2011, which is incorporated herein by reference in its entirety for all purposes.

### FIELD OF THE INVENTION

The invention relates generally to composite matrices for use in bone repair applications.

### BACKGROUND OF THE INVENTION

More than one million orthopedic operations are performed in the United States each year for reconstructive surgery, trauma, or abnormal skeletal defect (Pramer, A., et al., (eds): Musculoskeletal Conditions in the United States, Rosemont, Ill.: American Academy of Orthopaedic Surgeons, 1999, p. 34-39). The standard technique utilized to facilitate the arthrodesis process is the application of autogenous bone graft. Iliac crest graft harvesting is currently the ideal choice, as it is the only material that contains the three essential formation elements, which are osteogenic activity, osteoinductive and osteoconductive properties (Salgado, A. J., Macromolecules Bioscience 4: 743-765, 2004). Approximately 340,000 patients undergo iliac crest graft harvesting procedure each year. However, autogenous bone grafting comes with a significant cost. Bone graft harvesting is associated with significant clinical morbidity in terms of pain, scarring, increased surgical time, prolonged hospitalization, delayed rehabilitation, increased blood loss, increased infection risk, and surgical complications (i.e. fracture, hematoma, neuroma etc.). A review of the literature reveals that complications arise in 31% of the procedures and 27% of the patients continue to feel pain at 24 months after surgery (Gupta, A. R., Intl. Medical Journal 8: 163-166, 2001). Often the quantity of available graft is suboptimal and requires augmentation with allograft.

As an alternative, large bulk allografts have been utilized for reconstructive needs and are the current gold standard, as no equivalent alternative exists (Aro, H. T., et al., Annals of Medicine 25: 403-412, 1993). Allografts must be processed or devitalized to avoid immune rejection, but this results in a loss of cellular activity and healing potential as compared to autografts (Stevenson, S., et al., Clin. Orthop. Relat. Res. 324: 66-74, 1996). Allograft bone procedures are also associated with high complication rates due to lack of graft incorporation, delayed union at junction site, inflammatory immune issues, and potential for infectious diseases (Norman-Taylor, F. H. and Villar, R. N., Journal of Bone and Joint Surgery, Series B 79: 178-180, 1997). In a series of 1100 non-pelvic massive cadaveric allografts, primarily for treatment of bone tumors, only 61% were successful (Mankin, H. J., Chir. Organi Mov. 88: 101-113, 2003). Complications of fracture and non-union were the principle problems due to limited new bone formation and neovascularization of the structural allografts. Secondary infection following operative procedure to repair the fracture or graft non-union occurred in 53 cases and reduced the success rate to less than 30% (Id.).

For these reasons, a need exists to develop and validate alternative processes capable of providing bone regeneration and repair while eliminating associated morbidity and complications.

### SUMMARY OF THE INVENTION

A biocompatible and biodegradable composite bone matrix capable of supporting cell and tissue growth having at least one electrospun or solvent-cast synthetic polymer having nanoceramics uniformly dispersed throughout the polymer and a method of making the bone matrix are presented.

The composite bone matrix is prepared by the steps of

(a) combining a poly(α-hydroxy acid) polymer with a solvent methylene chloride for electrospinning or a solvent 1,1,1,3,3,3-hexafluoro-2-propanol for solvent-casting to form a solution, wherein the concentration of the poly(α-hydroxy acid) polymer ranges from 5-30%;

(b) combining a poly(α-hydroxy acid) polymer with the solvent methylene chloride for the electrospinning method or 1,1,1,3,m3,3-hexafluoro-2-propanol for the solvent-casting method to form a solution, wherein the concentration of the poly(α-hydroxy acid) polymer ranges from 5-30%;

(c) electrospinning the solution of (b) to form a fibrous matrix or

(d) applying the solution of (b) to a mold to form a non-fibrous matrix; and (e) freeze-drying the matrix of step (c) or step (d) to provide a dried matrix;

wherein the matrix is capable of supporting cell and tissue growth.

Methods for repairing a bone defect in a vertebrate subject are also presented. A first method involves the steps of

(a) introducing the composite bone matrix into a bone defect in a vertebrate subject; and

(b) allowing bone to regenerate within the bone defect.

A second method involves the steps of

(a) introducing the composite bone matrix into the interior of a section of graft bone;

(b) inserting the bone graft of step (a) into the bone defect; and

(c) allowing bone to regenerate within the bone defect.

A third method involves the steps of

(a) wrapping the composite bone matrix around the outside of a section of graft bone;

(b) inserting the bone graft of step (a) into the bone defect, and

(c) allowing bone to regenerate within the bone defect.

Each of these methods may further involve introducing whole bone marrow or isolated mesenchymal stem cells into the composite bone matrix before applying the composite bone matrix to the bone defect or graft bone.

### BRIEF DESCRIPTION OF THE DRAWINGS

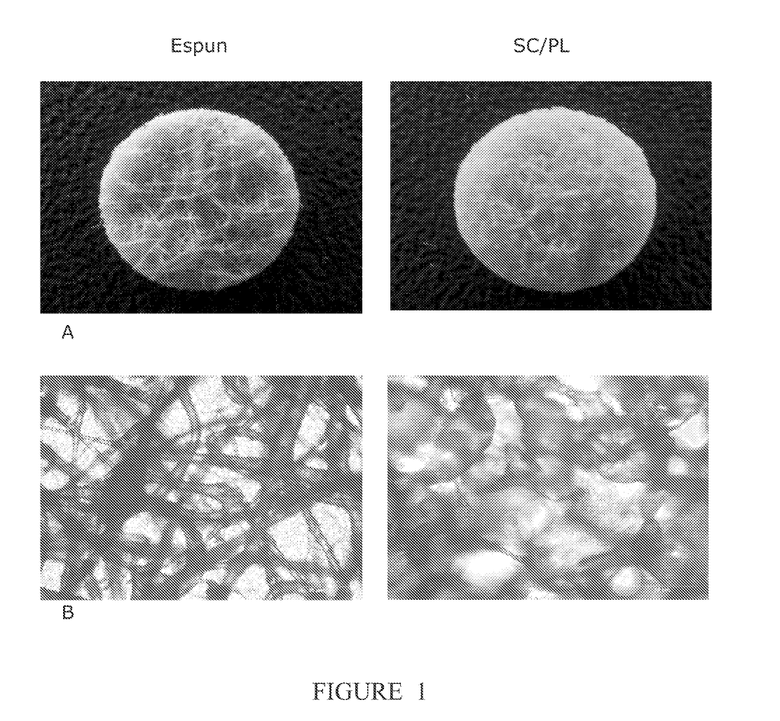

FIG. 1. Morphology of electrospun and solvent cast PCL composite mats after leaching
A. Gross morphology
B. Light microscopic morphology, bar=50 μm

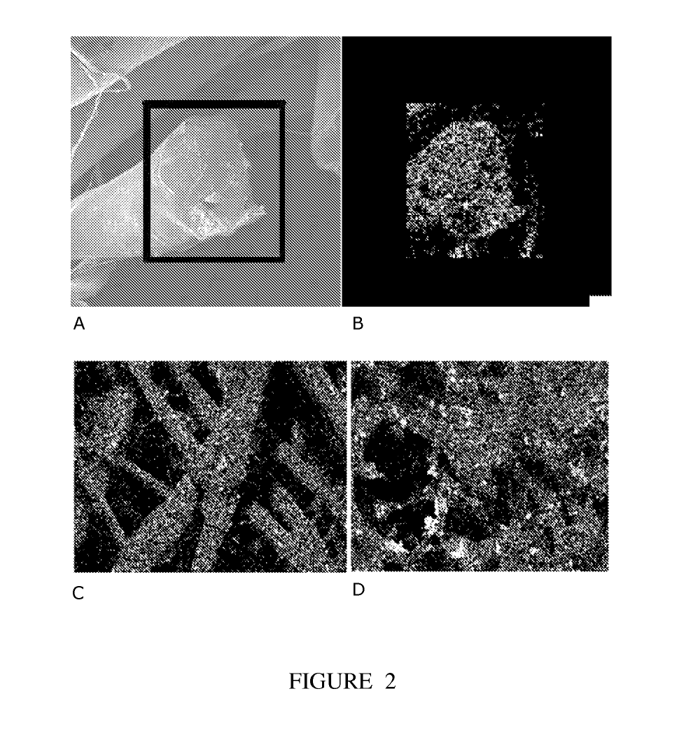

FIG. 2. A. Cross-section of electrospun PCL composite fiber
B. Electrospun fiber of A showing uniform distribution of ceramics within the fiber of A, imaged by energy dispersive X-ray analysis (EDXA) targeting calcium
C. Electrospun PCL composite fibers imaged by EDXA targeting potassium
D. Solvent-cast PCL composite fibers imaged by EDXA targeting potassium

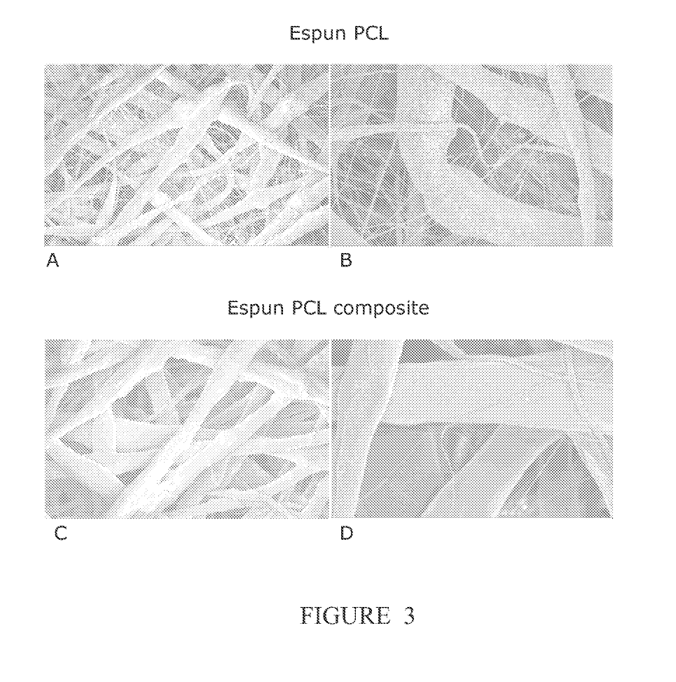

FIG. 3. Scanning electron micrographs of electrospun mats
A. PCL alone at 400× magnification
B. PCL alone at 15,000× magnification
C. PCL composite at 400×
D. PCL composite at 15,000× magnification.

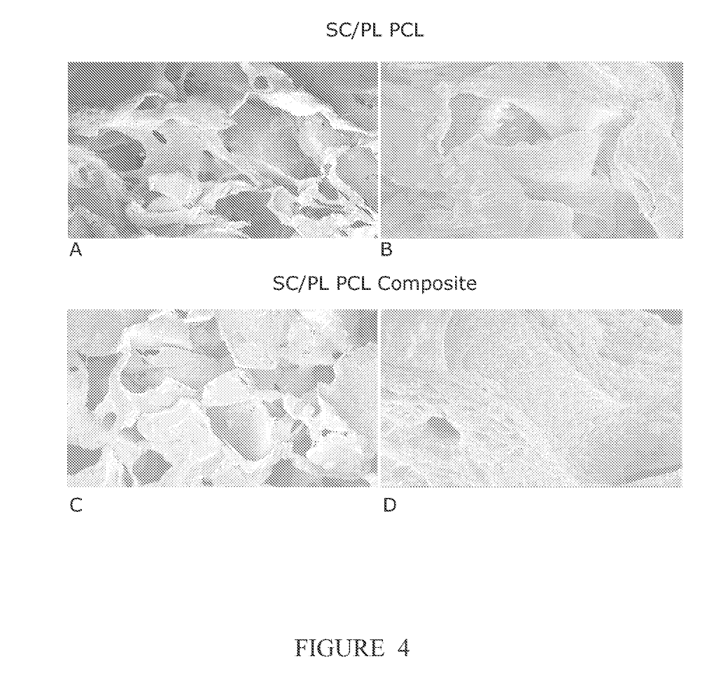

FIG. 4. Scanning electron micrographs of solvent cast mats
A. PCL alone+porogen leaching at 400× magnification
B. PCL alone+porogen leaching at 15,000× magnification
C. PCL composite+porogen leaching at 400×
D. PCL composite+porogen leaching at 15,000× magnification.

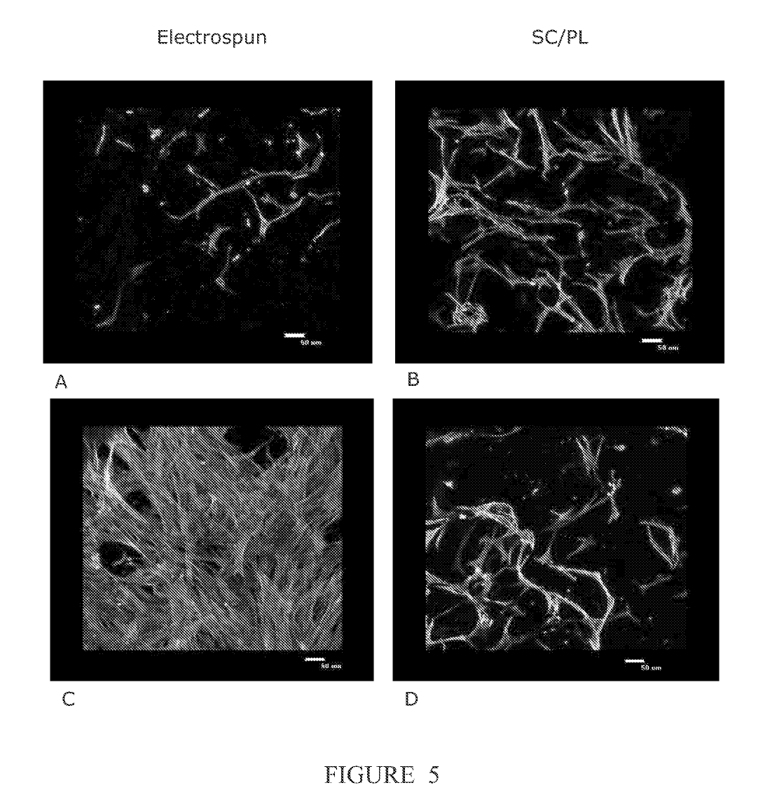

FIG. 5. Confocal images of MSCs cultured on PCL matrices, Day 7 of culture, bar=50 μm
A. Electrospun, porogen-leached, PCL alone matrix
B. Solvent-cast, porogen leached, PCL alone matrix
C. Electrospun PCL composite matrix
D. Solvent-cast, porogen leached, PCL composite matrix

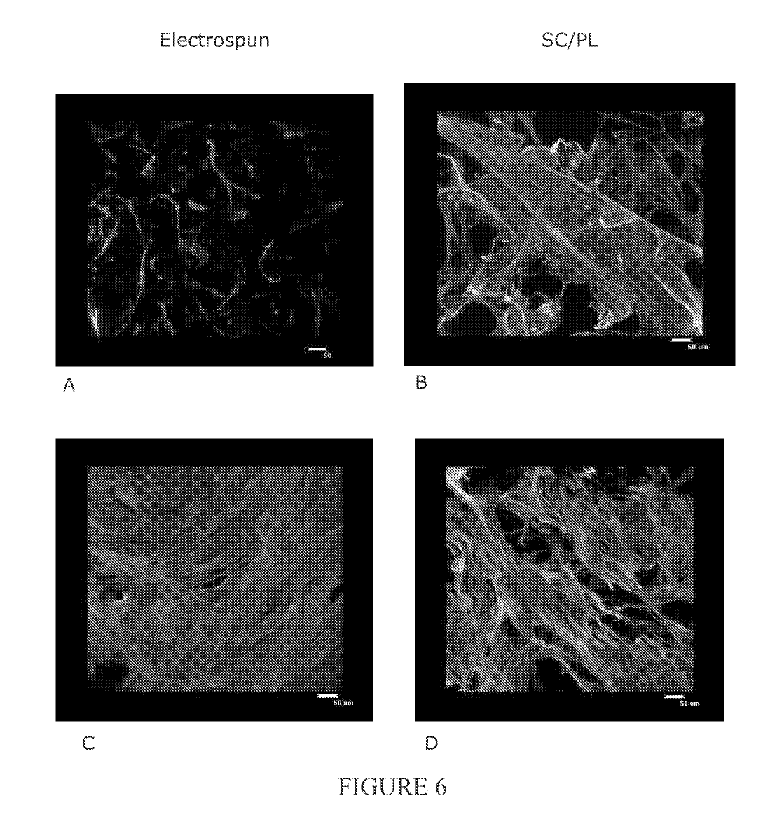

FIG. 6. Confocal images of MSCs cultured on PCL matrices, Day 14 of culture, bar=50 μm
A. Electrospun, porogen-leached, PCL alone matrix
B. Solvent-cast, porogen leached, PCL alone matrix
C. Electrospun PCL composite matrix
D. Solvent-cast, porogen leached, PCL composite matrix

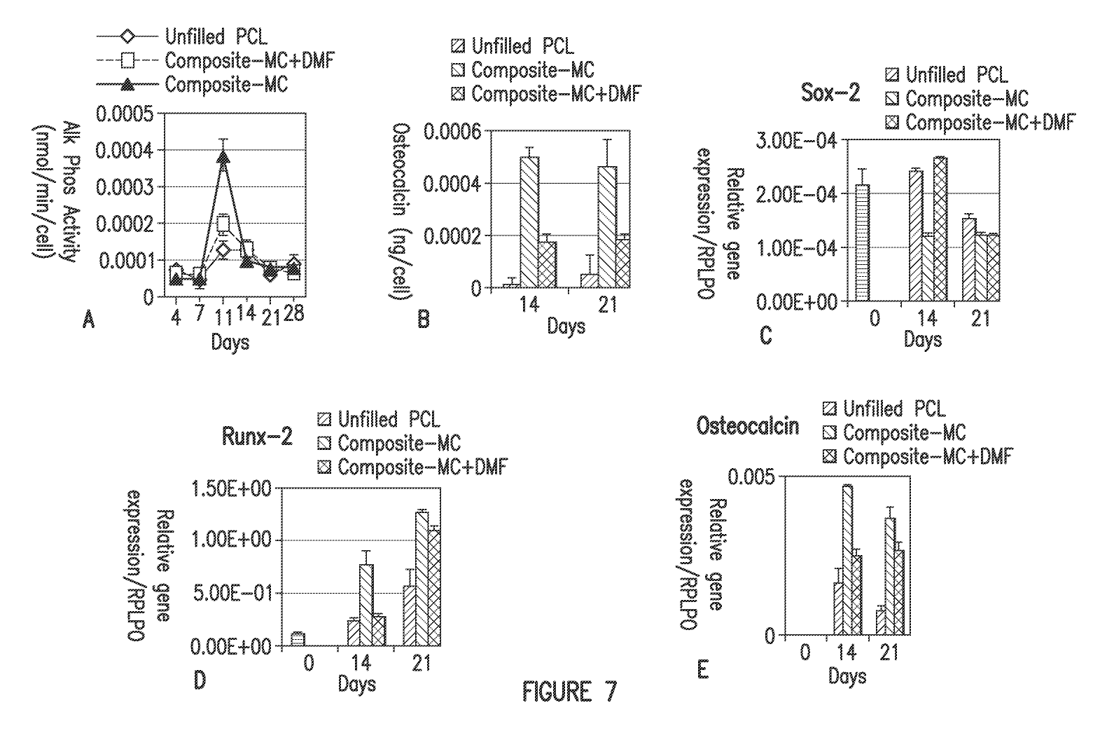

FIG. 7. Quantitative analysis of bone and stem cell markers of human MSCs on PCL matrices in standard growth medium. unfilled PCL=PCL alone; composite MC+DMF=PCL composite matrix having an average pore diameter<10 μm; composite MC=PCL composite matrix having pore diameters of about 150-250 μm.
A. Activity of alkaline phosphatase, marker for chondrogenesis
B. Osteocalcin protein levels, osteogenic marker
C. Expression of Sox-2, pluripotent cell marker
D. Expression of Runx-2, osteogenic cell marker
E. Expression of Osteocalcin, osteogenic cell marker

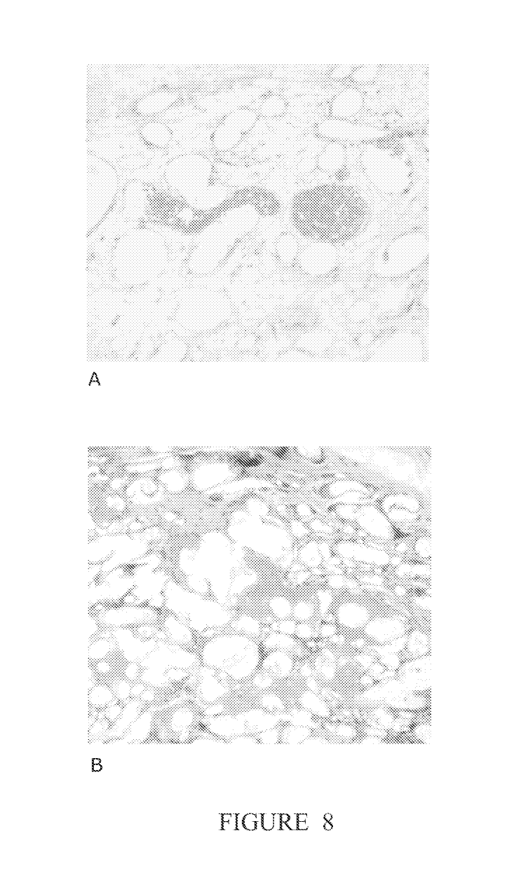

FIG. 8. Vascularization and bone formation in vivo on electrospun PCL and PLGA composite matrices eight weeks after implantation
A. Formation of blood vessels (red) by tissue on PCL composite.
B. Formation of bone and cartilage tissue (pink) on PLGA composite.

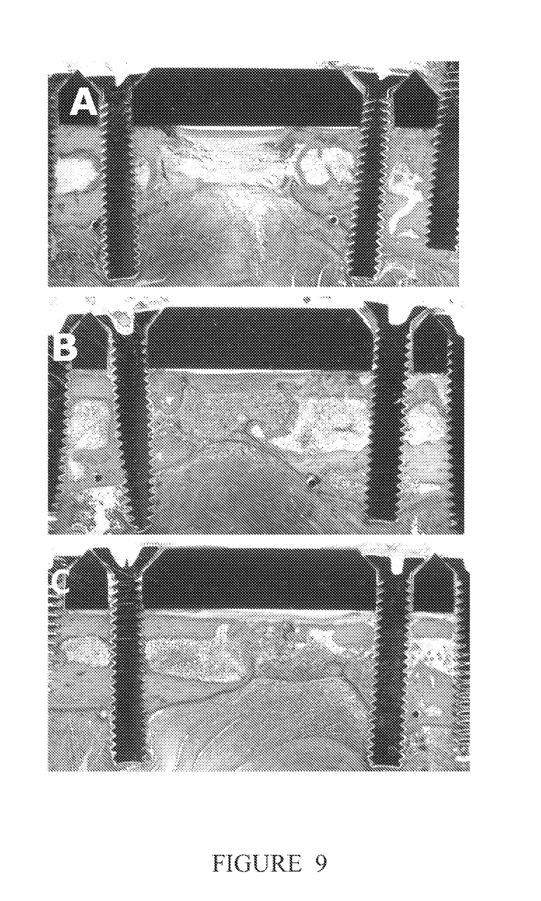

FIG. 9 New bone growth in rat segmental defect in vivo
A. Empty, untreated defect
B. PLGA composite matrix in defect
C. PLGA composite matrix plus whole bone marrow in defect

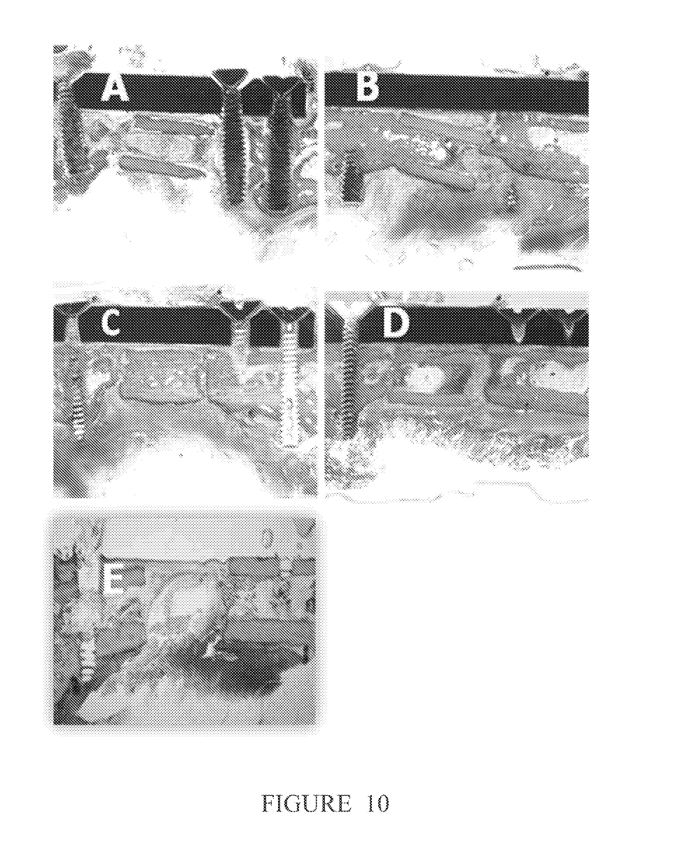

FIG. 10. New bone growth in vivo on allografts combined with PCL and PLGA composite matrices loaded with isolated MSCs eight weeks after implantation into rat segmental defects
A. PLGA composite inside allograft. New bone is pink.
B. PLGA composite wrapped around outside of allograft.
C. PCL composite inside allograft.
D. PCL composite wrapped around outside of allograft.
E. Empty defect (no allograft, no matrix). Host bone is orange.

### DETAILED DESCRIPTION OF THE INVENTION

Biocompatible, biodegradable, matrices for bone regeneration have been developed that can be utilized for periosteal and endosteal tissue engineered constructs. The matrices are designed to house mesenchymal progenitor cells and promote bone tissue formation. The composite matrices are composed of synthetic polymers containing bioactive nanoceramics and are mechanically flexible. The term “bioactive” refers to synthetic materials that form an interfacial bond with biological tissue upon implantation and enhance bone tissue formation as a result of surface modification when exposed to interstitial fluids. The matrices are entirely synthetic and do not induce an inflammatory response in the host subject. The matrices can be used alone or in combination with bone graft.

The composite matrices can be prepared with a single polymer or blended polymers. Suitable polymers include poly(α-hydroxy acids), such as the polyesters, polylactic acid (PLA), poly L-lactic acid (PLLA), polyglycolic acid (PGA), polylactic co-glycolic acid (PLGA), poly ε-caprolactone (PCL), poly methacrylate co-n-butyl methacrylate (PMMA), polydimethylsiloxane (PDMS), and polyethylene oxide (PEO). Polymer blending can be used to increase or decrease the degradation time of a matrix. For example, PCL degrades more slowly (1-1.5 years) than PGA (3 months) and the rate of degradation can be adjusted by using a blended combination of these polymers to form the matrix. The matrices have a final total concentration of polymer ranging from 5-30 wt %, preferably 10-25 wt %.

Nanoparticulate ceramics (nanoceramics) are present in the matrices at a final total concentration of 5-70 wt %, preferably 30-50 wt %, and range from 50-200 nm in diameter. Suitable nanoceramics include, but are not limited to, hydroxyapatite, tricalcium phosphate, biphasic calcium phosphate, calcium carbonate, calcium sulfate, bioactive glass, and biphasic bioceramics. A matrix may contain one or more nanoceramics. A preferred nanoceramic is the biphasic ceramic, hydroxyapatite/β-tricalcium phosphate (HA/βTCP), preferably at 20 HA/80 βTCP weight percent. Nanoceramics can be purchased or can be prepared according to known methods, e.g., Santos, G., et al., J. R. Soc. Interface, Jul. 18, 2012, Epublication.

Matrices are preferably produced from matrix solutions by electrospinning as described in Example 1A, or by solvent casting as described in Example 1B, using conventional methods, such as those described in Patlolla, A., at al., Acta Biomaterialia 6: 90-101, 2010, which is incorporated herein, in entirety, by reference. The polymer(s) is added to an appropriate solvent and the nanoceramic(s) is subsequently added to form a matrix solution. Appropriate solvents for specific polymers are known in the art and include methylene chloride (MC), 1,1,1,3,3,3-hexafluoro-2-propanol (HFIP), acetone, chloroform, dimethyl formamide (DMF), tetrahydrofuran (THF), and ethyl acetate. Preferred embodiments of the matrix solution include, but are not limited to, (1) 17 wt % PCL and 30 wt % HA/βTCP (20/80 wt %) in MC solvent; (2) 22 wt % PLGA (75% polylactide/25% gylcolic acid) and 30 wt % HA/μTCP (20/80 wt %) in MC solvent; (3) 17 wt % PCL and 30 wt % HA/βTCP (20/80 wt %) in HFIP solvent; and (4) 22 wt % PLGA (75/25) and 30 wt % HA/βTCP (20/80 wt %) in HFIP solvent. The matrix solution may also contain a non-ionic surfactant, e.g., Span® 80, or a cationic surfactant, e.g., cetyl trimethylammonium bromide (CTAB).

The matrices are prepared from the matrix solution by electrospinning or solvent casting. Electrospun matrices are fibrous, whereas solvent-cast matrices are sponge-like, as shown in FIG. 1. Formed matrices can be air dried or freeze-dried to remove remaining solvent. Freeze-drying is the more effective method. The dried matrices remain functional after storage under vacuum at room temperature for at least two years.

Matrix pore size and matrix porosity can be increased by adding particulate porogens to the matrix solution prior to electrospinning or solvent casting and then leaching the porogen out of the formed matrix, as described in Example 1. Pore size and matrix porosity can be modified by adjusting the size and concentration of the porogen used. Suitable porogens include inorganic salts such as CaCO₃ and NaCl, sugar crystals, such as saccharose, gelatin spheres and paraffin spheres.

The electrospun composite matrices exhibit fibers ranging from 100 nanometers to 100 micrometers in diameter. The fibers exhibit a uniform dispersion of the nanoceramic(s), as shown in FIG. 2, which enhances cellular attachment, infiltration and bone bioactivity. Interfiber spacing of the matrix ranges from 150 to 400 μm, preferably 150-250 μm. Interfiber spacing is determined, in part, by the polymer concentration. Matrix porosity ranges from 60 to 90%, preferably 80-85%. The Young's modulus of the composite matrix is similar to trabecular bone, and the matrix has an ultimate tensile strain of approximately 30%, demonstrating mechanical flexibility. Electrospun PCL and PCL composite matrices are shown in FIG. 3.

Solvent cast matrices subjected to porogen leaching also exhibit a uniform dispersion of the nanoceramic(s) and Young's modulus similar to that of electrospun matrices. Porosity of solvent cast matrices is also 60 to 90%, preferably 80-85%, and pore sizes range from 150-400 μm, preferably 150-250 μm. Solvent-cast, porogen leached (SC/PL) PCL and PCL composite matrices are shown in FIG. 4.

The matrices are useful for stimulating regeneration of bone tissue and repairing bone defects and may be used alone or in combination with whole bone marrow or isolated mesenchymal stem cells (MSCs). Bone marrow and MSCs may be isolated from the host subject or be acquired from donors. Methods for isolating MSCs are described in Example 2. Bone cell differentiation can be stimulated in vitro by adding whole bone marrow or isolated MSCs to the matrices and culturing in appropriate conditions, as described in Example 3 and shown in FIGS. 5-7.

The matrices are also useful for stimulating bone differentiation and repairing bone defects in vivo. Composite matrix may be implanted alone, or in combination with whole bone marrow or isolated MSCs, into a defect site as described in Example 4. The matrices stimulate blood vessel formation, bone and cartilage differentiation in vivo as shown in FIGS. 8 and 9.

In one embodiment, the matrices are used in combination with bone allografts to repair bone defects in vivo. The composite matrix can be cut to an appropriate size and can be shaped to fit inside the medullary canal lining the inner surface of the allograft or to wrap around the outside of the bone allograft, as described in Example 5. The composite matrix forms a contiguous interface with the allograft due to the ability of the nanoceramics to facilitate bonding of the material to bone, which may improve osteoconduction and integration of the allograft with the host bone. The composite matrix may be used alone or in combination with whole bone marrow or isolated MSCs. The matrix houses mesenchymal progenitor cells and promotes bone tissue formation, which is a primary characteristic of the natural periosteum and endosteum. When wrapped around the outside of the allograft, the composite matrix appears to act as a substitute periosteum and, when inserted inside the allograft, acts as a substitute endosteum. In both positions, the composite matrix promotes bone differentiation and tissue formation within the bone defect and in the endosteal and periosteal regions of the host bone, as shown in FIG. 10.

The composite matrices described above are characterized by pore sizes and/or interfiber spacing that allows cell infiltration and bone tissue in-growth, a maximum concentration of ceramic for improved bioactivity, and homogeneous dispersion of the ceramic in the fibers for improved molecular interaction and mechanical properties. The combination of synthetic polymers with ceramics provides mechanically flexible matrices that can easily be sized and shaped for use within bone defects and in combination with bone grafts to provide complete repair of bone defects and full return of function to the repaired bone.

The compositions and methods of this disclosure are described with reference to the following examples, however, these examples are provided for illustrative purposes only and are not limiting. Changes, modifications, enhancements and refinements to these compositions and methods may be made without departing from the spirit or scope of the disclosure and are encompassed by the invention.

### Examples

### 1. Preparation of Matrices

A. Composite Electrospun Fiber Matrices

Composite electrospun fibers were prepared by the methods of Patlolla, A., et al., Acta Biomaterialia 6: 90-101, 2010. Briefly, poly ε-caprolactone (PCL) or polylactide-co-glycolic acid (PLGA, 75% polylactide/25% glycolic acid) were combined with the ceramic, hydroxy-apatite/β-tricalcium phosphate (HA/βTCP, 20/80 wt %) in the solvent, methylene chloride. Polymer was present in the resulting solution at 17 wt % and HA/βTCP was present at 30 wt %. The solution was briefly sonicated and subjected to electrospinning to form fiber matrices (mats). The morphology of the electrospun mats is shown in FIG. 1. FIG. 2 demonstrates that the distribution of ceramics within the spun fibers is uniform.

For comparison, an electrospun matrix containing PCL alone was prepared having a similar porosity and pore size/interfiber spacing as the PCL composite. A solution of PCL alone was prepared. CaCO₃ with a particle size of 20 nm was added to this PCL solution as a particulate porogen to increase porosity and pore size/interfiber spacing of the matrix. After formation of the PCL mat, CaCO₃ was leached from the mats by treating the mats with 3M HCl for several hours. After leaching, the mats were washed with deionized water to remove excess acid and were air dried.

FIG. 3 presents scanning electron micrographs of espun, PCL-leached (A,B) and PCL composite (C,D) mats. Both PCL-leached mats and PCL composite mats exhibited a bimodal distribution of fibers of both micron and sub-micron dimensions, which favors cell infiltration. In both types of mats, interfiber spacing was about 200-250 μM, and porosity was about 79%.

B. Composite Solvent Cast Matrices

Conventional methods for solvent casting, e.g., as described by Kim, et al., S. S., Biomaterials 27: 1399-1409, 2006, were used to prepare the matrices. Briefly, PCL was combined with HA/3TCP in the solvent, 1,1,1,3,3,3-hexafluoro-2-propanol (HFIP) at 10% v/v PCL and 30 wt % HA/βTCP. NaCl (90% v/v) with a particle size of 200-500 μM was added to the solution as a particulate porogen. The solution was mixed by stirring and poured onto a glass/aluminum mold. The solvent was removed by freeze-drying at −40° C. overnight. NaCL was subsequently leached from the resulting mats with water. After leaching, the mats were treated with increasing concentrations of ethanol up to 100% and then air dried.

FIG. 4 presents scanning electron micrographs of solvent-cast, porogen-leached, PCL (A,B) and PCL composite (C,D) mats. In both types of mats pore size averaged about 300 μm and porosity was about 84%.

### 2. Preparation of Mesenchymal Stem Cells

MSCs were obtained from human whole bone marrow (healthy male donors, 18-35 years old), purchased from Lonza, Inc. Rat and Human MSCs were isolated from the bone marrow and processed according to previously published protocols. (Arinzeh, T. L., et al., Biomaterials 26: 3631-3638, 2005; Breitbart, E. A., et al., J. Orthopaedic Research 28: 942-949, 2010; Livingston, T., et al., J Biomed. Mat. Res. 62: 1-13, 2002; Haynesworth, S., et al., J. Cell Physiology 138: 8-16, 1992). Briefly, two donors per species were used for in vitro studies. MSCs from both species were isolated and cultured in standard growth media (Dufbecco's Modified Eagle's Medium (DMEM) containing 10% fetal bovine serum and 1% antibiotic for human and alpha-MEM with 15% fetal bovine serum and 1% antibiotic for rat). MSCs were characterized for multipotency by performing osteogenesis, chondrogenesis and adipogenesis assays. MSCs were characterized by flow cytometry for MSC surface antigens (positive for CD44 and CD29 and negative for CD14, CD45, and CD34) and were used at passage 2 for all in vitro differentiation studies.

3. Osteogenic and Chondrogenic Differentiation of MSCs on Composite Matrix in vitro

Discs with a thickness of 0.3 mm and diameter of 6 mm were prepared from composite electrospun or solvent-cast matrices, prepared as described in Example 1, sterilized with 100% ethanol, and air dried. Discs were placed in 96 well plates and MSCs prepared as described in Example 2 were seeded on each disc at about 10,000 cells/well as previously described in Arinzeh, T. T., et al., Biomaterials 26: 3631-3638, 2005. Cells were cultured in humidified incubators at 37° C./5% CO₂. After 7 or 14 days of culture, discs were fixed with 4% formaldehyde, washed and stained for cytoskeleton actin filaments with ALEXA FLUOR® 488 phalloidon (Invitrogen Molecular Probes) and with 4′-6-diamidino-2-phenylindole (DAPI) (Invitrogen Molecular Probes) for cell nuclei.

Confocal microscopy of the cells on day 7 and day 14 is shown in FIGS. 5 and 6, respectively. Electrospun composite fiber mats (FIG. 5C) appeared to initially provide more rapid attachment and growth than solvent-cast mats (FIGS. 5B,D) or espun, leached PCL mats (FIG. 5A), but cell density on electrospun and solvent-cast composite mats was similar at 14 days (FIG. 6). The espun, leached, PCL only mat showed poor cell attachment and growth at both 7 and 14 days. Cells infiltrated the full depth of the matrix for both electrospun and solvent cast mats (data not shown).

Gene expression associated with bone differentiation was examined quantitatively by real-time RT-PCR. Human MSCs were cultured on electrospun PCL composite matrices having an average pore size of about 150-250 um (Composite-MC), PCL alone prepared without porogens (unfilled PCL), or a PCL composite matrix having a pore size<10 um (Composite-MC+DMF). Quantitative RT-PCR analysis was performed with the One Step QUANTITECT® SYBR® Green RT-PCR Kit (Qiagen and Life Technologies, respectively) using the MX4000™ detection system (Stratagene), according to the methods described by Weber, N., et al., Acta Biomaterialia 6: 3550-3556, 2010. Briefly, total RNA was isolated from cell samples using the RNEASY® Mini Kit (Qiagen) including the homogenization (QIAshredder) and DNA digestion step (RNase Free DNase Set). Reverse transcription was conducted for 30 min at 50° C., followed by PCR activation for 15 min at 95° C. Forty amplification cycles were run, consisting of 15 s denaturation at 94° C., 30 s of annealing at 55° C., and 30 s of extension at 72° C.

Results are shown in FIG. 7. Composite mats having larger pore sizes provided superior induction of bone cell differentiation. Sox-2, a marker for undifferentiated MSC, declined as gene expression of osteocalcin and Runx-2, bone cell markers, increased with time in culture in the absence of added osteoinductive factors in the culture media (FIGS. 7 C,D,E). These results demonstrate that the composite mats stimulate bone cell differentiation in vitro. Cells on the composite matrices also demonstrated high levels of alkaline phosphatase activity (FIG. 7A), a chondrocyte hypertrophic marker, and production of osteocalcin (FIG. 7B) after 11 and 14 days of culture, respectively, in standard culture medium without the presence of inductive factors. These results further confirm that the composite mats are capable of promoting osteogenesis.

4. Bone Repair on Composite Matrix in vivo

Human and rat MSCs were loaded onto the matrices as described above and evaluated for bone differentiation and tissue formation by implanting the matrices subcutaneously in the backs of Severe Combined Immunodeficient (SCID) mice according to previously reported protocols (Arinzeh, T. T., et al., Biomaterials 26: 3631-3638, 2005). Briefly, a 1-cm transverse incision was made along the dorsum of the back of the animal. Blunt dissection was performed to separate the skin from the subcutaneous connective tissues and form several pockets under the skin into which the implants were inserted. Comparisons were made with implanted matrices alone (without MSCs) eight weeks after implantation.

Results are shown in FIGS. 8 and 9. The composite matrices showed extensive bone and blood vessel formation at 8 weeks of implantation in ectopic sites when loaded with hMSCs (FIG. 8B, PLGA composite). Blood vessel formation was also detected in matrices that were not loaded with hMSCs (FIG. 8A, PCL composite). FIG. 9 shows serial sections of PLGA composite matrix in rat segmental defects at 8 weeks after implantation. In the absence of any matrix, little or no new bone growth was observed within the defect (FIG. 9A). PLGA composite matrix alone stimulated new bone growth within the defect (FIG. 9B). PLGA composite matrix in combination with whole bone marrow stimulated new bone growth that crossed the entire defect by 8 weeks (FIG. 9C). These results demonstrate that the composite matrices are useful, alone or in combination with bone marrow or isolated MSCs, for repairing bone defects and can be used as substitutes for bone grafts.

5. Bone Repair on Graft Bone Combined with Composite Matrix In Vivo

Rat, femoral segmental defects were treated with the composite matrices with or without MSCs in combination with an allograft. This defect model is well established and has been described in Azad, V., et al., J. Orthopaedic Trauma 23: 267-276, 2009; and Breitbart, E. A., et al., J. Orthopaedic Research 28: 942-949, 2010. This model was chosen because it is a load-bearing, critical sized defect which results in a non-union if left untreated. Male, Fisher 344 rats approximately 80 days old were utilized for these experiments. Allograft bone was obtained from the femurs of the treated Fisher 344 rats. A 5 mm bone section was removed from the diaphysis of the right femur and was cleaned, processed and γ-irradiated prior to use.

Femoral segmental defects were created unilaterally and treated with bulk allograft associated with electrospun PCL composite and PLGA composite matrices loaded with isolated MSCs. For insertion in the defect site, the composite matrix was wrapped around the outer surface of the graft (periosteum), or placed inside the graft adjacent to the inner surface (endosteum). After eight weeks, transverse serial sections of the grafted regions were prepared, stained with hematoxylin and eosin, and observed by light microscopy. Serial sections were analyzed to determine the area of new bone formed within the defects.

Results are shown in FIG. 10. Both PCL composite (FIGS. 10A,B) and PLGA composite (FIGS. 10C,D) matrices enhanced bone differentiation and repair of the bone defect when inserted within (FIGS. 10A,C) or wrapped around the allograft (FIGS. 10B,D). Bone formation occurred in the endosteal and periosteal regions of the host bones and within the defect for all composite matrices. The greatest amount of new bone was produced by allografts wrapped with PLGA composite matrices (4.24 mm² bone area; compared with 2.55 mm² area for PLGA composite inside allograft; 2.95 mm² area for PCL composite inside allograft; 2.71 mm² area for PCL composite wrapped around allograft).

An untreated, empty defect (FIG. 10E) showed no new bone formation. These results demonstrate that the composite matrices stimulate new bone formation and enhance the incorporation of allografts in vivo.

We claim:

1. A biocompatible and biodegradable composite bone matrix capable of supporting cell and tissue growth comprising at least one electrospun or solvent-cast synthetic polymer comprising nanoceramics uniformly dispersed throughout the polymer.

2. The composite bone matrix of claim 1, wherein the nanoceramics have a diameter of 50-200 nanometers.

3. The composite bone matrix of claim 1, wherein the porosity of the matrix is 60 to 90 percent.

4. The composite bone matrix of claim 1, wherein the porosity of the matrix is 80 to 85 percent.

5. The composite bone matrix of claim 1, wherein the matrix comprises at least one electrospun polymer forming a plurality of electrospun fibers, the fibers having diameters ranging from 100 nanometers to 100 micrometers.

6. The composite bone matrix of claim 1, wherein the composite bone matrix comprises at least one electrospun synthetic polymer, the matrix having an interfiber spacing of 150 to 400 micrometers.

7. The composite bone matrix of claim 6, wherein the matrix has an interfiber spacing of 150 to 250 micrometers.

8. The composite bone matrix of claim 1, wherein the matrix comprises at least one solvent-cast synthetic polymer, the matrix having pore sizes of 150 to 400 micrometers.

9. The composite bone matrix of claim 8, wherein the matrix has pore sizes of 150 to 250 micrometers.

10. The composite bone matrix of claim 1, wherein the at least one synthetic polymer is a poly(α-hydroxy acid) polymer.

11. The composite bone matrix of claim 10, wherein the poly(α-hydroxy acid) is selected from the group consisting of polylactic acid, poly L-lactic acid, polyglycolic acid, polylactic co-glycolic acid, poly ε-caprolactone, poly methacrylate co-n-butyl methacrylate, poly dimethyl siloxane, and polyethylene oxide.

12. The composite bone matrix of claim 1, wherein the at least one ceramic is selected from the group consisting of hydroxy apatite, tricalcium phosphate, biphasic calcium phosphate, calcium carbonate, calcium sulfate, bioactive glass, and biphasic bioceramic.

13. The composite bone matrix of claim 8, wherein the ceramic is the biphasic bioceramic hydroxyapatite/β-tricalcium phosphate.

14. A method of preparing the composite bone matrix of claim 1 comprising the steps of
(a) combining a poly(α-hydroxy acid) polymer with a solvent methylene chloride for electrospinning or a solvent 1,1,1,3,3,3-hexafluoro-2-propanol for solvent-casting to form a solution, wherein the concentration of the poly(α-hydroxy acid) polymer ranges from 5-30%;
(b) adding to the solution of (a) a ceramic selected from the group consisting of hydroxy apatite, tricalcium phosphate, biphasic calcium phosphate, calcium carbonate, calcium sulfate, bioactive glass, and biphasic bioceramic, wherein the concentration of ceramic ranges from 5-70%; and
(c) electrospinning the solution of (b) to form a fibrous matrix or
(d) applying the solution of (b) to a mold to form a non-fibrous matrix; and
(e) freeze-drying the matrix of step (c) or step (d) to provide a dried matrix;
wherein the matrix is capable of supporting cell and tissue growth.

15. The method of claim 14 further comprising adding a porogen to the solution of step b and removing the porogen from the dried matrix of step (e) by leaching.

16. A method for repairing a bone defect in a vertebrate subject comprising the steps of
(a) introducing the composite bone matrix of claim 1 into a bone defect in a vertebrate subject; and
(b) allowing bone to regenerate within the bone defect.

17. The method of claim 16 further comprising introducing whole bone marrow or isolated mesenchymal stem cells into the composite bone matrix before introducing the composite bone matrix into the bone defect.

18. A method for repairing a bone defect in a vertebrate subject comprising the steps of
(a) introducing the composite bone matrix of claim 1 into the interior of a section of bone graft;
(b) inserting the bone graft of step (a) into the bone defect; and
(c) allowing bone to regenerate within the bone defect.

19. The method of claim 18 further comprising introducing whole bone marrow or isolated mesenchymal stem cells into the composite bone matrix before introducing the composite bone matrix into the section of donor bone.

20. A method for repairing a bone defect in a vertebrate subject comprising the steps of
(a) wrapping the composite bone matrix of claim 1 around the outside of a section of bone graft;
(b) inserting the bone graft of step (a) into the bone defect, and
(c) allowing bone to regenerate within the bone defect.

21. The method of claim 20 further comprising introducing whole bone marrow or isolated mesenchymal stem cells into the composite bone matrix before wrapping the composite bone matrix around the section of donor bone.
